(19) United States
(12) Patent Application Publication
(10) Pub. No.: US 2007/0156038 A1
(43) Pub. Date: Jul. 5, 2007

(54) Method to image the heart

(76) Inventors: Herbert D. Zeman, Memphis, TN (US); Gunnar Lovhoiden, Bartlett, TN (US); Dana Capocaccia, Germantown, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 11/652,419

(22) Filed: Jan. 11, 2007

Related U.S. Application Data
(63) Continuation-in-part of application No. 10/386,249, filed on Mar. 11, 2003.
Application No. 10/386,249 is a continuation-in-part of application No. 09/487,007, filed on Jan. 19, 2000, now Pat. No. 6,556,858.

(51) Int. Cl. A61B 5/00; G06F 19/00

(52) U.S. Cl. 600/315; 702/19

(57) ABSTRACT

A method to enhance the visibility of the vascular structure of the heart is disclosed. The method uses the previous imaging system to illuminate the heart with a wavelength of light this is adsorbed by blood. Using this material, we can differentiate the difference between the concentrations of hemoglobin within the vessels, the heart muscle, and in the chambers of the heart. A viable light image of the vascular structure of the heart is coaxially project on to the heart. It is contemplated that this method could assist a surgeon in coronary heart surgery.

### CROSS REFERENCE TO RELATED APPLICATIONS

This application is a continuation-in-part of U.S. Pat. No. 6,556,858 filed Jan. 19, 2000, entitled Imaging System Using Diffuse Infrared Light and U.S. Ser. No. 10/386,249 filed Mar. 11, 2003 entitled Imaging System Using Diffuse Infrared Light (hereby specifically incorporated by reference in their entirety).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

### BACKGROUND OR THE INVENTION

The present invention is generally directed to a system and method for illuminating an object with infrared light and producing a video image of the object based on reflected infrared light. The previously described imaging system can be used to image the vascular structure of the heart.

### BRIEF SUMMARY OF THE INVENTION

The invention provides methods to enhance the visibility of the vascular structure of the heart. This can be accomplished by illuminating the heart with infrared light; producing a visible light image of the vascular structure of the heart and coaxially projecting an enhanced visible light image on to the surface of the heart.

### BRIEF DESCRIPTION OF THE SEVERAL VIEWS OF THE DRAWINGS

Further advantages of the invention will become apparent by reference to the detailed description of preferred embodiments when considered in conjunction with the drawings, which are not to scale, wherein like reference characters designate like or similar elements throughout the several drawings as follows:

The patent or application file contains at least one drawing executed in color. Copies of this patent or patent application publication with color drawing(s) will be provided by the Office upon request and payment of the necessary fee.

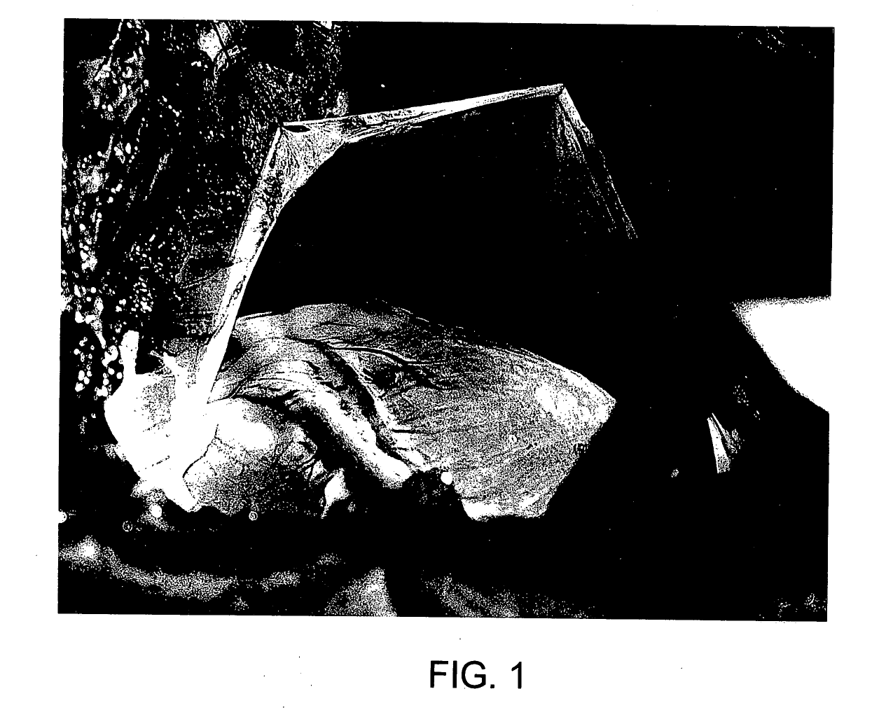

FIG. 1 is a photograph of a surgically exposed pig heart.

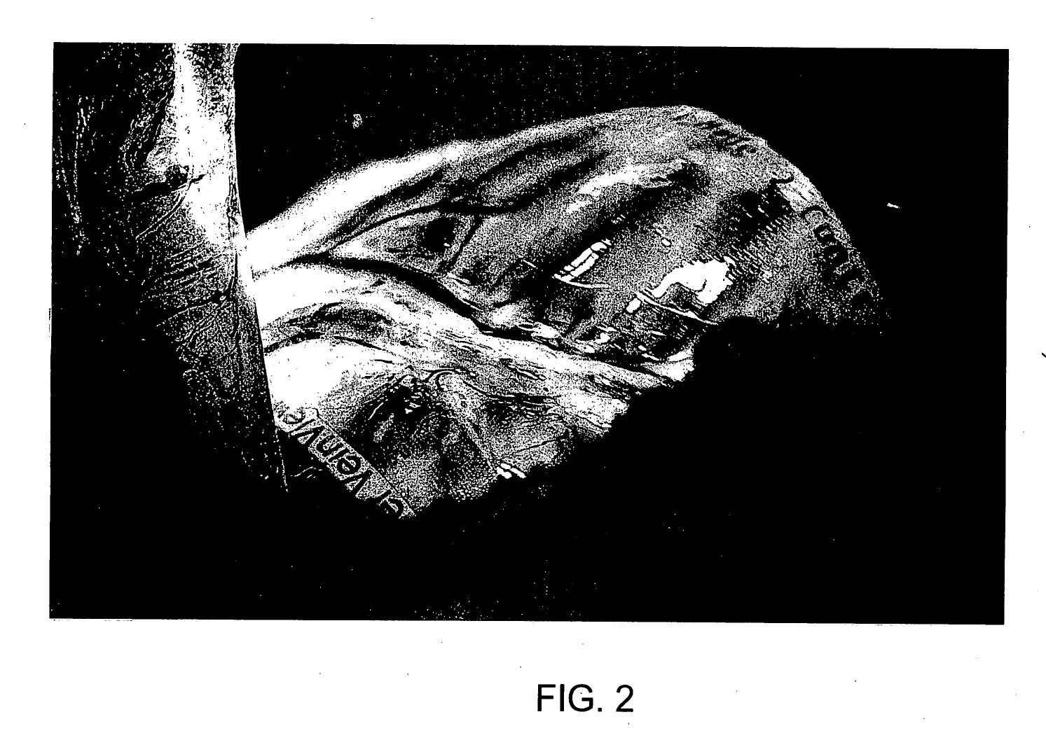

FIG. 2 is a photograph with a visible light image of the vascular structure projected on to the heart.

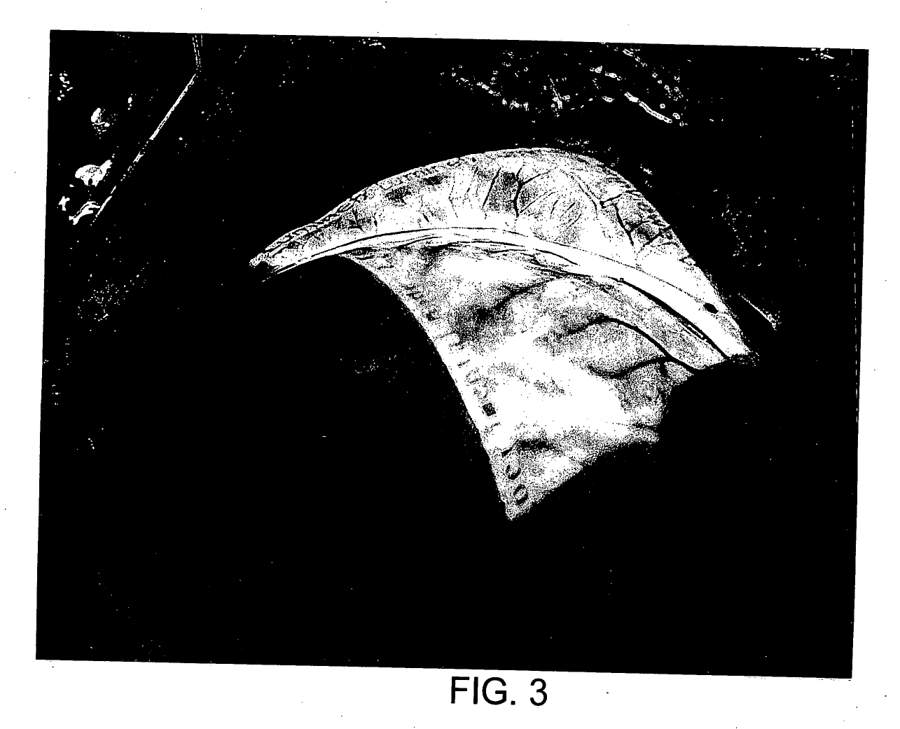

FIG. 3 is a photograph with a visible light image of the vascular structure projected on to the heart.

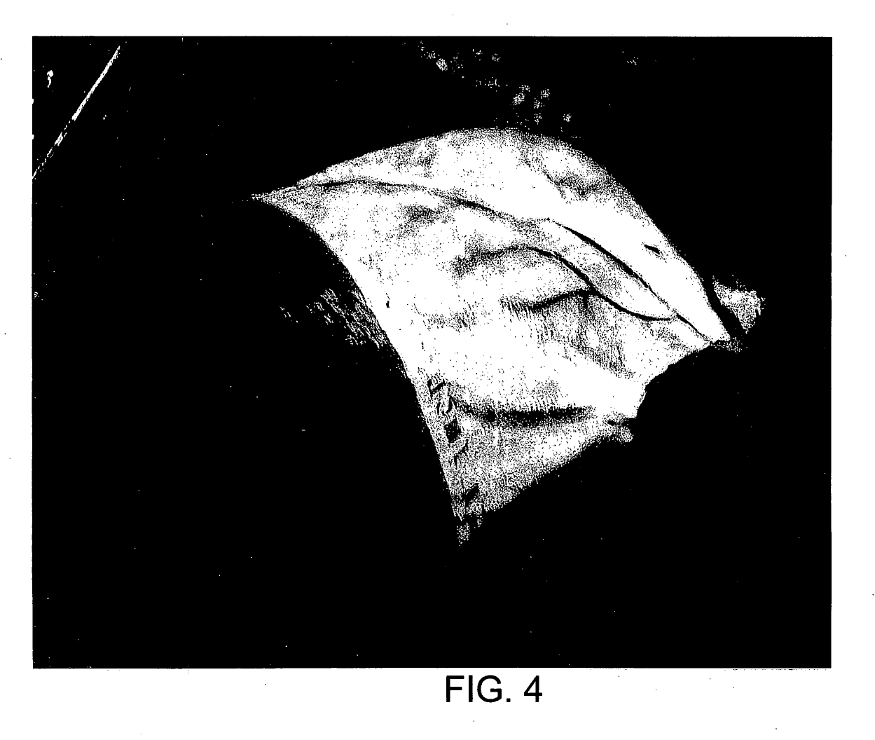

FIG. 4 is a photograph with a visible light image of the vascular structure projected on to the heart.

### DETAILED DESCRIPTION OF THE INVENTION

Skin and some other body tissues reflect infrared light while blood absorbs radiation. Thus, in video images of body tissue taken under infrared illumination, blood vessels appear as dark lines against a lighter background of surrounding flesh.

The human heart is covered by a fat pad that completely obstructs a doctor's view of the coronary arteries, when just looking at a heart. The current method of performing coronary artery bypass surgery is to essentially fillet that fat pad very slowly off the heart until the doctor gets to the point where he can find the coronary artery and perform the necessary surgery on it. In this procedure, it is possible to nick the coronary artery during the process of removing fat. The present apparatus can be used to show the vessel on the heart when surgery is about to be performed and will project an image of the underlying coronary artery and other heart blood vessels on top of the fat pad so that a doctor who needs to perform surgery on any of those coronary vessels can remove only the fat necessary to access the vessel that he is attempting to repair. The surgeon does not need to remove excess fat or potentially cause any excess blood loss. The method works while the heart is beating or while the heart is arrested.

The surface of the heart can be illuminated by infrared light. In an alternative embodiment, such as, heart valve replacement surgery, the interior of the heart can be illuminated to facilitate surgery. In this procedure, the illuminated heart produces a spectural pattern that is detected by a detector, such as, a CCD detector. The detector transmits a raw image of the vascular structure to a computer for enhanced processing to produce a visible light image of the vascular structure of the heart. The computer enhanced vasculator structure is coaxially projected onto the surface of the heart in one embodiment, or in the alternative embodiment into the interior of the heart. Coaxially projection means that the spectural pattern produced by illuminating the heart and detected by the detector is coaxially with the projected visible light image. The coaxially projection results in vascular image overlying the heart surface in an embodiment, or in the alternative embodiment, overlying the internal heart structure, close to an internal structure such as a valve.

In FIG. 1, a pig heart is exposed by surgery and photographed to show how a heart appears during a surgical procedure. FIGS. 2-4 show a visible light image of the vascular structure of the heart. The dark bands are arteries in the heart. Knowledge of the location of vascular structure, such as coronary arteries in the heart is beneficial to the surgeon during heart surgery.

### EXAMPLE 1

A 100 lb pig was anesthetized and placed on ventilator support. A sternotomy was performed. The chest was opened exposing the pericardium. The pericardial sac was entered revealing a small beating heart. The left anterior descending coronary artery was easily identified. An infra-red light was placed over the pericardial well. As shown in FIGS. 1-4, the light revealed a clear distinction between the coronary arteries and the coronary veins. The arteries appeared light green while the veins appeared black. The infra-red light showed bubbles in the coronary arteries that could be seen fluctuating in the arterial blood flow. These bubbles could not be seen in the ambient light.

Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

We claim:

1. A method to enhance the visibility of vascular structure of a heart comprising:
a) exposing the heart;
b) illuminating the heart with a wavelength of light that is adsorbed by blood;
c) producing a visible light image of said vascular structure on said heart; and
d) coaxially projecting a computer enhanced visible light image of said vascular structure on to said heart.

2. The method of claim 1 wherein said heart is beating.

3. The method of claim 1 wherein said heart is arrested.
